(19) United States
(12) Patent Application Publication
(10) Pub. No.: US 2007/0237712 A1
(43) Pub. Date: Oct. 11, 2007

(54) Methods of Redistributing Apical Target Antigens to Detect and Treat Cellular Proliferative Disease

(76) Inventors: Ayyappan K. Rajasekaran, Los Angeles, CA (US); Jason J. Christiansen, Los Angeles, CA (US)

Correspondence Address: BEYER WEAVER LLP, P.O. BOX 70250, OAKLAND, CA, 94612-0250, US

(21) Appl. No.: 11/628,877

(22) PCT Filed: Jun. 14, 2005
(86) PCT No.: PCT/US05/20983

§ 371 Date: Dec. 8, 2006

Related U.S. Application Data
(60) Provisional application No. 60/679,905, filed on May 10, 2005.
(60) Provisional application No. 60/579,955, filed on Jun. 14, 2004.

(51) Int. Cl. A61K 51/10; A61K 31/407; A61K 31/70; G01N 33/577; A61K 39/395

(52) U.S. Cl. 424/1.49; 424/143.1; 424/9.1; 435/7.21; 514/25; 514/413; 514/42

(57) ABSTRACT

Methods of detecting and treating a cellular proliferative in a subject through the use of an antibody and a compound that disrupts the apical trafficking of a target antigen are disclosed. In a one embodiment, the target antigen is PSMA.

This application claims the benefit of priority of U.S. Application Ser. No. 60/579,955, filed Jun. 14, 2004, and U.S. Application Ser. No. 60/679,905, filed May 10, 2005, the entire contents of which are incorporated herein by this reference.

### STATEMENT REGARDING FEDERALLY SPONSORED RESEARCH OR DEVELOPMENT

This invention was made with government support under grant DAMD17-02-1-0661 awarded by the Department of Defense and grant DK56216 awarded by the National Institutes of Health. The government has certain rights in the invention.

### FIELD OF THE INVENTION

The present invention relates to the treatment of a cellular proliferative disease characterized by the presence of a target antigen with apical polarity.

### BACKGROUND OF THE INVENTION

In order to gain access to antigens on the surface of malignant cells in vivo, therapeutic monoclonal antibodies (mAbs) must traverse a gamut of formidable obstacles. With few barriers to impede mAb binding, hematological malignancies are well suited to this form of therapy (42). In comparison, successful treatment of solid tumors with mAbs has proven more difficult to obtain. Studies using radiolabled mAbs demonstrate that only 0.01%-0.1% of the original injected dose, will ever reach antigen within a solid tumor mass, per gram of tissue (43, 44). Following intravenous injection and diffusion throughout the vascular space, therapeutic antibodies must traverse the microvascular endothelium and contend with stromal and interstitial barriers associated with a sizeable tumor mass (45, 46). After navigating these impediments, a mAb may still be confronted by an additional set of epithelial barriers that may severely restrict accessibility of antigens to circulating antibodies. While over 90% of all cancers are carcinomas derived from epithelial tissues, the significance of these epithelial barriers is often disregarded in the treatment of malignant disease.

Although often overlooked, epithelial barriers may exert a profound impact on the efficacy of mAb therapy. The tight junctions would severely restrict the accessibility of antibodies to antigens at the apical plasma membrane. For example, the carcinoembryonic antigen (CEA), which is expressed at similar levels in both benign and malignant cells of the colonic epithelium, is restricted to the apical surface of normal tissues and well-differentiated tumors (47). However, loss of tight junction integrity in poorly differentiated tumors results in non-polarized expression of CEA throughout the plasma membrane (47, 48). The altered localization of CEA allows accessibility of this antigen to the underlying vasculature and would explain why immunoscintigraphic studies using intravenously injected mAbs to CEA are able to specifically label primary and metastatic tumors, but not normal or well-differentiated tissues (49, 50).

Prostate specific membrane antigen (PSMA) is a 100 kDa transmembrane glycoprotein with a highly restricted profile of tissue expression. In addition to the benign prostatic epithelium, PSMA is expressed in tumor-associated neovasculature and at increased levels in most cases of prostate cancer, with the greatest levels associated with high-grade tumors, metastases, and androgen independent disease (1-4). Overexpression of PSMA is a good prognostic indicator of disease outcome and PSMA is more highly expressed in poorly differentiated tumors than the better known marker, prostate specific antigen (PSA) (4).

PSMA is an attractive marker for the development of PSMA-targeted prostate cancer therapeutics and diagnostics. Monoclonal antibodies against PSMA have shown high affinity and specificity for prostate cancer cells in vitro and in mouse models. However, due to the apical polarity of PSMA, there are delivery problems with immunotherapy using antibodies specific for PSMA.

Accordingly, there exists a need for new approaches to optimize the use of target antigens, such as PSMA, which exhibit apical polarity for use in the cellular proliferative disease.

### SUMMARY OF THE INVENTION

The present invention provides for methods of detecting and treating cellular proliferative diseases where the diseases are characterized by the presence of a target antigen with apical polarity.

One aspect of the invention provides for a method of treatment of a cellular proliferative disease in a subject, wherein the disease is characterized by the presence of a target antigen with apical polarity. The method comprises administering to the subject a compound that disrupts the apical trafficking of the target antigen and an antibody specific for the target antigen. In a preferred embodiment, the cellular proliferative disease is a cancer. Prostrate cancer is one such example of a cancer treatable by this embodiment.

In a preferred embodiment of this aspect, the target antigen is PSMA. In a further preferred embodiment the antibody is specific for PSMA. Examples of anti-PSMA antibodies include 7E11, J591 and PM2J004.5.

In one embodiment of this aspect, the compound administered to the subject disrupts microtubule integrity; preferably the compound is a vinca alkaloid. Examples of suitable vinca alkaloids include vinblastine, vincristine, vindesine and vinorelbine.

In another embodiment of this aspect, the compound administered to the subject interferes with N-glycosylation of the target antigen. Examples of suitable compounds include tunicamycin, swainsonine, and deoxymannojirimycin.

In yet another embodiment of this aspect, the antibody further comprises an effector group. In a preferred embodiment, the effector group is a cytotoxic agent. Examples of suitable cytotoxic agents include radioisotopes, radionuclides, and chemotherapeutic agents.

Another aspect of the invention provides for a method of detecting the presence of a cellular proliferative disease in a subject, wherein the disease is characterized by the presence of a target antigen with apical polarity. The method comprises administering to the subject a compound that disrupts the apical trafficking of the target antigen, administering to the subject an antibody specific for the target antigen, and detecting the binding of the antibody to the target antigen. In a preferred embodiment, the cellular proliferative disease is a cancer. Prostrate cancer is one such example of a cancer treatable by this embodiment.

In a preferred embodiment of this aspect, the target antigen is PSMA. In a further preferred embodiment the antibody is specific for PSMA. Examples of anti-PSMA antibodies include 7E11, J591 and PM2J004.5.

In one embodiment of this aspect, the compound administered to the subject disrupts microtubule integrity; preferably the compound is a vinca alkaloid. Examples of suitable vinca alkaloids include vinblastine, vincristine, vindesine and vinorelbine.

In another embodiment of this aspect, the compound administered to the subject interferes with N-glycosylation of the target antigen. Examples of suitable compounds include tunicamycin, swainsonine, and deoxymannojirimycin.

In yet another embodiment of this aspect, the antibody further comprises a detectable label. Examples of suitable detectable labels include radioisotopes, radionuclides, fluorescent groups, paramagnetic groups, enzymatic groups, chemiluminescent groups, biotinyl groups, or predetermined polypeptide epitopes recognized by a secondary reporter.

Other aspects of the invention provide for methods of detecting the presence of a cellular proliferative disease in a sample wherein the disease is characterized by the presence of a target antigen with apical polarity. In one embodiment, the method comprises contacting the sample with a compound that disrupts the apical trafficking of the target antigen, contacting the basolateral membrane of the sample with an antibody specific for the target antigen, and detecting the binding of the antibody to the target antigen on the basolateral membrane. In another embodiment, the method comprises administering to the subject a compound that disrupts the apical trafficking of the target antigen, removing a sample from the subject, contacting the basolateral membrane of the sample with an antibody specific for the target antigen, and detecting the binding of the antibody to the target antigen basolateral membrane.

### DESCRIPTION OF THE DRAWINGS

FIG. 1A depicts immunofluorescence analysis of tissue sections showing PSMA localization at the apical plasma membrane of polarized epithelial cells. FIG. 1B depicts surface immunofluorescence analysis performed on confluent monolayers of MDCK cells expressing PSMA (MDCK-PSMA) showing prominent PSMA localization at the apical plasma membrane. FIG. 1C depicts results from three independent cell surface biotinylation assay demonstrating that 70 to 79% of PSMA at the cell surface was localized to the apical plasma membrane. The scale bars in FIGS. 1A and 1B represent 1 um.

FIGS. 2A and 2B show that the majority of PSMA is targeted directly to the apical plasma membrane. FIG. 2C depicts a selective cell surface biotinylation-based targeting assay demonstrating that PSMA is seen predominantly on the apical plasma membrane with a smaller fraction localized to the basolateral surface. Error bars indicate standard deviation.

FIG. 3A shows that a GFP tagged form of PSMA in which the majority of the extracellular domain was removed (PSMA-D103-750) is localized in a non-polarized fashion. FIG. 3B shows an immunoblot analysis demonstrating that 90-95% of the α-subunit of the sodium pump (Na,K-ATPase α-sub) was localized at the basolateral surface of these cells. FIG. 3C demonstrates that a secreted form of PSMA, sPSMA, is secreted almost exclusively from the apical plasma membrane. Error bars indicate standard deviation of three independent experiments.

FIG. 4 depicts that inhibition of N-glycosylation by treatment with tunicamycin abolished the polarized expression of PSMA and resulted in equivalent levels at both plasma membrane surfaces.

FIG. 5A depicts cell surface biotinylation experiments, which demonstrate an homogeneous distribution of PSMA at both plasma membrane domains following nocodazole treatment. FIG. 5B shows that polarity of the basolateral marker Na,K-ATPase was unaffected by nocodazole treatment. FIG. 5C shows that polarized delivery of PSMA is abolished following three hours of treatment with nocodazole.

FIG. 6A depicts an immunofluorescence analysis of alpha-tubulin showing intact microtubules in untreated control cells. Extensive microtubule depolymerization is observed following treatment with the vinca alkaloids vinblastine (FIG. 6B), vincristine (FIG. 6C), and vinorelbine (FIG. 6D). FIG. 6E shows that MDCK-PSMA cells readily internalize mAb J591 added to the apical chamber. FIG. 6I shows that very little mAb J591 is internalized from the basolateral surface. Following treatment with the vinca alkaloids vinblastine (FIG. 6F), vincristine (FIG. 6G), and vinorelbine (FIG. 6H), J591 was also taken up from the apical surface. These cells exhibited a dramatic increase in J591 internalization from the basolateral surface (FIGS. 6J-L). The scale bars in FIGS. 6A-L represent 10 um.

FIG. 7A depicts a histological assessment of a metastatic lesion from lymph nodes showing diffused prostrate tumor infiltration replacing the lymph node parenchyma. Small areas where tumor cells are forming acinar/glanduar-appearing structures are indicated by arrows. FIG. 7B depicts a high magnification image of the boxed section of FIG. 7A showing well-differentiated epithelial tissue. FIGS. 7C and D depict immunohistochemical analysis using anti-PSMA antibodies showing polarized expression of PSMA on the apical plasma membrane of some of the glandular structures. The arrows indicate PSMA staining in endothelial cells surrounding small lymphovascular lumens. FIGS. 7E and F depict immunohistochemical analysis showing that endothelial cells surrounding lymphvacular lumens stain positively for CD34.

### DETAILED DESCRIPTION OF THE INVENTION

The present invention provides for methods of detecting and treating a cellular proliferative disease where the disease is characterized by the presence of a target antigen with apical polarity. The methods comprise disrupting the trafficking of a target antigen to the apical surface. The disruption results in a redistribution of the target antigen to the basolateral plasma membrane where the antigen is more accessible to the delivery of agents via the vasculature. The methods further comprise the use of antibodies specific for the redistributed antigens in the detection and treatment of the disease.

All epithelial tissues is comprised of highly polarized cells with biochemically distinct apical and basolateral plasma membrane surfaces (7). These plasma membrane domains maintain an asymmetrical distribution of proteins and lipids and are physically separated by tight junctions (TJs) that promote cell-cell contact, restrict the flow of fluid through inter-cellular spaces, and prevent the lateral diffusion of membrane components (8, 9). Thus, apical and basolateral plasma membrane domains are exposed to disparate extracellular environments. While the basolateral plasma membrane is relatively accessible to the underlying vasculature, TJs prevent molecules within the circulation from reaching the apical surface (6). The tight junctions severely restrict the accessibility of antibodies to antigens at the apical plasma membrane.

Accordingly, it is one aspect of this invention to provide a method of disrupting the trafficking of the target antigen to the apical surface. In one embodiment, the trafficking is disrupted by disrupting the integrity of cytoskeletal elements, such as microtubules. As cells establish polarity, microtubules emanating from the microtubule organizing centers are rearranged to form longitudinal arrays with their minus ends facing the apical surface (20). This polarized arrangement of microtubules is involved in trafficking of a number of apical proteins, since microtubule depolymerization or disruption of dynein function results in aberrant trafficking of several apical proteins to the basolateral surface (21, 22), but does not appear to have a significant impact on trafficking of basolateral proteins (21, 23).

In a preferred embodiment microtubule integrity is disrupted by treatment with a chemotherapeutic agent. In a further preferred embodiment, microtubule integrity is disrupted by treatment with nocodazole (methyl[5-(2-thienyl-carbonyl)-1H-benzimidazol-2-yl]-carbamate). In yet a further preferred embodiment, microtubule integrity is disrupted by treatment with a vinca alkaloid such as vinblastine, vincristine, or vinorelbine. Disruption of microtubule integrity results in redistribution of apical target antigens from the apical surface to the basolateral plasma membrane where they are more accessible to agents delivered via the vasculature.

In further embodiment, the trafficking of the target antigen to the apical surface is disrupted by a compound that interferes with N-glycoslyation of the target antigen. The trafficking of antigens to the apical surface of a cell is mediated by an array of signals including N- or O-linked oligosaccharides (14, 15). Interference with glycoslyation of the target antigen disrupts the trafficking of the antigen to the apical surface of the cell. As shown in the examples, treatment of a cell with a compound that interferes with N-glycosylation of an antigen results in redistribution of the antigen from the apical surface to the basolateral plasma membrane. In a preferred embodiment, the compound that interferes with N-glycoslyation of the target antigen is tunicamycin. In a further preferred embodiment, the compound that interferes with N-glycoslyation of the target antigen is swainsonine. In a further preferred embodiment, the compound that interferes with N-glycoslyation of the target antigen is deoxymannojirimycin.

In one embodiment, the target antigen is the prostate specific membrane antigen (PSMA). PSMA is a 100 kDa transmembrane glycoprotein expressed on the surface of the prostatic epithelium. In addition to the benign prostatic epithelium, PSMA is expressed in tumor-associated neovasculature and at increased levels in most cases of prostate cancer, with the greatest levels associated with high-grade tumors, metastases, and androgen independent disease (1-4). Additionally, PSMA is expressed in the neovasculature associated with cancers such as, for example, conventional (clear cell) renal cell, transitional cell of the bladder, testicular-embryonal, neuroendocrine, colon, and breast cancer. (Chang, S., Advances in Prostrate Cancer, 6 (10): 13-18).

In one embodiment, the invention provides for a method of treating a cellular proliferative disease in a subject, where the disease is characterized by the presence of a target antigen with apical polarity, comprising administering to a subject a compound that disrupts the apical trafficking of the target antigen and an antibody specific for the target antigen. Redistribution of the target antigen from the apical surface of the cell to the basolateral membrane allows for increased accessibility of the antigen to antibodies delivered via the vasculature.

The term “subject” includes human and animal subjects.

As used herein, the term “antibody” refers to a monomeric or multimeric protein comprising one or more polypeptide chains. An antibody can bind specifically to an antigen and may be able to inhibit or modulate the biological activity of the antigen. In certain embodiments, antibodies are produced by recombinant DNA techniques. In additional embodiments, antibodies are produced by enzymatic or chemical cleavage of naturally occurring antibodies. Antibodies include, but are not limited to, F(ab), F(ab′), F(ab′)2, Fv, and single chain Fv fragments, as well as single-chain, chimeric, humanized, fully human, polyclonal, and monoclonal antibodies (mAb). At a minimum, an antibody, as meant herein, comprises a polypeptide that can bind specifically to an antigen comprising all or part of a light or heavy chain variable region.

A variable region comprises at least three heavy or light chain CDRs, see, supra (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342: 877-883), embedded within a framework region (designated framework regions 1-4, FR1, FR2, FR3, and FR4, by Kabat et al., 1991, supra; see also Chothia and Lesk, 1987, supra). See, infra.

Traditional antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2.

Within light and heavy chains, the variable and constant regions are joined by a “J” region of about twelve (12) or more amino acids, with the heavy chain also including a “D” region of about ten (10) more amino acids. See, generally, Paul, W., ed., 1989, Fundamental Immunology Ch. 7, 2nd ed. Raven Press, N.Y. The variable regions of each light/heavy chain pair form the antibody binding site.

The variable regions of the heavy and light chains typically exhibit the same general structure of relatively conserved framework regions (FR) joined by three hypervariable regions, also called complementarily determining regions or CDRs. The CDRs are the hypervariable regions of an antibody that are responsible for antigen recognition and binding. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest. Chothia et al., 1987, J. Mol. Biol. 196:901-917; Chothia et al., 1989, Nature 342:878-883.

CDRs constitute the major surface contact points for antigen binding. See, e.g., Chothia and Lesk, 1987, J. Mol. Biol. 196:901-917. Further, CDR3 of the light chain and, especially, CDR3 of the heavy chain may constitute the most important determinants in antigen binding within the light and heavy chain variable regions. See, e.g., Chothia and Lesk, 1987, supra; Desiderio et al., 2001, J. Mol. Biol. 310:603-615; Xu and Davis, 2000, Immunity 13:37-45; Desmyter et al., 2001, J. Biol. Chem. 276:26285-26290; and Muyldermans, 2001, J. Biotechnol. 74:277-302. In some antibodies, the heavy chain CDR3 appears to constitute the major area of contact between the antigen and the antibody. Desmyter et al., 2001, supra. In vitro selection schemes in which CDR3 alone is varied can be used to vary the binding properties of an antibody. Muyldermans, 2001, supra; Desiderio et al., 2001, supra.

In one embodiment, the antibody is a monoclonal antibody (mAb), with from one (1) to six (6) of the depicted CDRs, as outlined herein. The antibodies of the invention may be of any type including IgM, IgG (including IgG1, IgG2, IgG3, IgG4), IgD, IgA, or IgE antibody. In preferred embodiment, the antibody is an IgG type antibody. In an even more preferred embodiment, the antibody is an IgG2 type antibody.

In some embodiments, the CDRs are all from the same species, e.g., human. Alternatively, CDRs may be either from other species (e.g., murine CDRs), or may be different human CDRs than those depicted in the sequences. For example, human CDRH3 and CDRL3 regions from the appropriate sequences identified herein may be used, with CDRH1, CDRH2, CDRL1 and CDRL2 being optionally selected from alternate species, or different human antibody sequences, or combinations thereof. For example, the CDRs of the invention can replace the CDR regions of commercially relevant chimeric or humanized antibodies.

In some embodiments, the antibody may be a chimeric antibody and/or a humanized antibody. In general, both “chimeric antibodies” and “humanized antibodies” refer to antibodies that combine regions from more than one species. For example, “chimeric antibodies” traditionally comprise variable region(s) from a mouse (or rat, in some cases) and the constant region(s) from a human. “Humanized antibodies” generally refer to non-human antibodies that have had the variable-domain framework regions swapped for sequences found in human antibodies. Generally, in a humanized antibody, the entire antibody, except the CDRs, is encoded by a polynucleotide of human origin or is identical to such an antibody except within its CDRs. The CDRs, some or all of which are encoded by nucleic acids originating in a non-human organism, are grafted into the beta-sheet framework of a human antibody variable region to create an antibody, the specificity of which is determined by the engrafted CDRs. The creation of such antibodies is described in, e.g., WO 92/11018, Jones, 1986, Nature 321:522-525, Verhoeyen et al., 1988, Science 239:1534-1536. Humanized antibodies can also be generated using mice with a genetically engineered immune system. Roque et al., 2004, Biotechnol. Prog. 20:639-654. In the present invention, the identified CDRs are human, and thus both humanized and chimeric antibodies in this context include some non-human CDRs; for example, humanized antibodies may be generated that comprise the CDRH3 and CDRL3 regions, with one or more of the other CDR regions being of a different special origin.

Antibodies of the present invention also include antibody fragments. Specific antibody fragments include, but are not limited to, (i) the Fab fragment consisting of VL, VH, CL and CH1 domains, (ii) the Fd fragment consisting of the VH and CH1 domains, (iii) the Fv fragment consisting of the VL and VH domains of a single antibody; (iv) the dAb fragment (Ward et al., 1989, Nature 341:544-546) which consists of a single variable, (v) isolated CDR regions, (vi) F(ab′)₂ fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., 1988, Science 242:423-426, Huston et al., 1988, Proc. Natl. Acad. Sci. U.S.A. 85:5879-5883), (viii) bispecific single chain Fv dimers (PCT/US92/09965) and (ix) “diabodies” or “triabodies”, multivalent or multispecific fragments constructed by gene fusion (Tomlinson et. al., 2000, Methods Enzymol. 326:461-479; WO94/13804; Holliger et al., 1993, Proc. Natl. Acad. Sci. U.S.A. 90:6444-6448). The antibody fragments may be modified. For example, the molecules may be stabilized by the incorporation of disulphide bridges linking the VH and VL domains (Reiter et al., 1996, Nature Biotech. 14:1239-1245). Again, as outlined herein, the non-CDR components of these fragments are preferably human sequences.

In preferred embodiments, the target antigen is PSMA and the antibody is specific for PSMA. Examples of antibodies specific for PSMA include 7E11, J591 and PM2J004.5. Liu, H., et al., Cancer Res, 58: 4055-4060 (1998).

“Specifically binds” or “binds specifically to” as used herein means the antibody:antigen equilibrium dissociation constant is <10⁻⁷ to 10⁻¹⁰ M, more preferably <10⁻⁸ to <10⁻¹⁰ M, even more preferably <10⁻⁹ to <10⁻¹⁰ M.

In some embodiments, the antibodies of the invention comprise the addition of one or more labels. Examples of such labels include labelling groups and effector groups.

The term “labelling group” means any detectable label. Examples of suitable labelling groups include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent groups (e.g., FITC, rhodamine, lanthamide phosphors), paramagnetic groups, enzymatic groups (e.g., horseradish peroxidase, β-galactosidase, luciferase, alkaline phosphatase), chemiluminescent groups, biotinyl groups, or predetermined polypeptide epitopes recognized by a secondary reporter (e.g., leucine zipper pair sequences, binding sites for secondary antibodies, metal binding domains, epitope tags). In some embodiments, the labelling group is coupled to the antibody via spacer arms of various lengths to reduce potential steric hindrance. Various methods for labelling proteins are known in the art and may be used in performing the present invention.

The term “effector group” means any group coupled to an antibody that acts as a cytotoxic agent. Examples for suitable effector groups are radioisotopes or radionuclides (e.g., ³H, ¹⁴C, ¹⁵N, ³⁵S, ⁹⁰Tc, ⁹⁹Tc, ¹¹¹In, ¹²⁵I, ¹³¹I). Other suitable groups include toxins, therapeutic groups, or chemotherapeutic groups. Examples of suitable groups include calicheamicin, auristatins, geldanamycin and maytansine. In some embodiments, the effector group is coupled to the antibody via spacer arms of various lengths to reduce potential steric hindrance.

In general, labels fall into a variety of classes, depending on the assay in which they are to be detected: a) isotopic labels, which may be radioactive or heavy isotopes; b) magnetic labels (e.g., magnetic particles); c) redox active moieties; d) optical dyes; enzymatic groups (e.g. horseradish peroxidase, β-galactosidase, luciferase, alkaline phosphatase); e) biotinylated groups; and f) predetermined polypeptide epitopes recognized by a secondary reporter (e.g., leucine zipper pair sequences, binding sites for secondary antibodies, metal binding domains, epitope tags, etc.). In some embodiments, the labelling group is coupled to the antibody via spacer arms of various lengths to reduce potential steric hindrance. Various methods for labelling proteins are known in the art and may be used in performing the present invention.

Preferred labels include optical dyes, including, but not limited to, chromophores, phosphors and fluorophores, with the latter being preferred in many instances. Fluorophores can be either “small molecule” fluores, or proteinaceous fluores.

By “fluorescent label” is meant any molecule that may be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade BlueJ, Texas Red, IAEDANS, EDANS, BODIPY FL, LC Red 640, Cy 5, Cy 5.5, LC Red 705, Oregon green, the Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE) (Molecular Probes, Eugene, Oreg.), FITC, Rhodamine, and Texas Red (Pierce, Rockford, Ill.), Cy5, Cy5.5, Cy7 (Amersham Life Science, Pittsburgh, Pa.). Suitable optical dyes, including fluorophores, are described in Molecular Probes Handbook by Richard P. Haugland, hereby expressly incorporated by reference.

Suitable proteinaceous fluorescent labels also include, but are not limited to, green fluorescent protein, including a Renilla, Ptilosarcus, or Aequorea species of GFP (Chalfie et al., 1994, Science 263:802-805), EGFP (Clontech Laboratories, Inc., Genbank Accession Number U55762), blue fluorescent protein (BFP, Quantum Biotechnologies, Inc. 1801 de Maisonneuve Blvd. West, 8th Floor, Montreal, Quebec, Canada H3H 1J9; Stauber, 1998, Biotechniques 24:462-471; Heim et al., 1996, Curr. Biol. 6:178-182), enhanced yellow fluorescent protein (EYFP, Clontech Laboratories, Inc.), luciferase (Ichiki et al., 1993, J. Immunol. 150:5408-5417), f, galactosidase (Nolan et al., 1988, Proc. Natl. Acad. Sci. U.S.A. 85:2603-2607) and Renilla (WO92/15673, WO95/07463, WO98/14605, WO98/26277, WO99/49019, U.S. Pat. Nos. 5,292,658, 5,418,155, 5,683,888, 5,741,668, 5,777,079, 5,804,387, 5,874,304, 5,876,995, 5,925,558). All of the above-cited references are expressly incorporated herein by reference.

Accordingly, one aspect of the invention provides for a method of treatment of a cellular proliferative disease in a subject, wherein the disease is characterized by the presence of a target antigen with apical polarity. The method comprises administering to the subject a compound that disrupts the apical trafficking of the target antigen and an antibody specific for the target antigen wherein the antibody is labeled with an effector group. In preferred embodiment, the effector group is a cytotoxic agent. In a further preferred embodiment, the cytotoxic agent is selected from among radioisotopes, radionuclides, and chemotherapeutic agents.

Another aspect of the invention provides for methods of detecting the presence of a cellular proliferative disease in a subject, where the disease is characterized by the presence of a target antigen with apical polarity. In one embodiment of this aspect, the method comprises administering to a subject a compound that disrupts the apical trafficking of the target antigen and an antibody specific for the target antigen. The binding of the antibody to the target antigen is then detected using any suitable method.

For diagnostic applications, the antibody typically will be labeled with a detectable labeling group. Suitable labeling groups include, but are not limited to, the following: radioisotopes or radionuclides (e.g., 3H, ¹⁴C, ¹⁵N, ³⁵S, 90Y, ⁹⁹Tc, ¹¹¹In, ¹²⁵I, ¹³¹I), fluorescent groups (e.g., FITC, rhodamine, lanthamide phosphors), paramagnetic groups, enzymatic groups (e.g., horseradish peroxidase, β-galactosidase, luciferase, alkaline phosphatase), chemiluminescent groups, biotinyl groups, or predetermined polypeptide epitopes recognized by a secondary reporter (e.g., leucine zipper pair sequences, binding sites for secondary antibodies, metal binding domains, epitope tags). In some embodiments, the labelling group is coupled to the antibody via spacer arms of various lengths to reduce potential steric hindrance. Various methods for labelling proteins are known in the art and may be used in performing the present invention.

This aspect can be used to detect, diagnose, or monitor diseases and/or conditions which are characterized by the presence of a target antigen with apical polarity either in vivo or in vitro.

In vivo detection of a target antigen can be performed using methods known in the art. For example, in vivo detection can be performed using scintigraphic imaging or magnetic resonance imaging (MRI).

If scintigraphic imaging is to be used, the labelling group will comprise a radioisotope. Suitable isotopes include gamma-emitting isotopes such as 1-131, 1-123, Tc-99m, In-111 and Ga-67. Scanning is effected with either a conventional planar and/or SPECT gamma camera, or by use of a hand held gamma probe used externally or internally to localize the tumor, microbiological site of infection, myocardial infarct, atherosclerotic plaque or other target site. Scanning can also be effected by any method known in the art. The scintigram is normally taken by a gamma imaging camera having one or more windows for detection of energies in the 50-500 keV range. Use of radioisotopes with high energy beta or positron emissions would entail use of imaging cameras with the appropriate detectors, all of which are conventional in the art.

If magnetic resonance imaging (MRI) is to be used, the labelling group will comprise a MRI enhancing species. Examples of labelling groups useful for MRI image enhancement include paramagnetic Gd(III), Eu(III), Dy(III), Pr(III), Pa(IV), Mn(II), Cr(III), Co(III), Fe(III), Cu(II), Ni(II), Ti(III) and V(IV) ions, or radicals, e.g., nitroxides. These paramagnetic groups would be conjugated to a substrate bearing paramagnetic ion chelators for the ions or linkers for the radical addends. The image enhancing agent must be present in sufficient amounts to enable detection by an external camera, using magnetic field strengths which are reasonably attainable and compatible with patient safety and instrumental design. MRI-basic principles and applications, 2^{nd} ed. Wiley-Liss, Inc. (1999), incorporated by reference herein.

In vitro detection of a target antigen can be performed using immunohistological methods known to those of skill in the art (e.g., Tijssen, 1993, Practice and Theory of Enzyme Immunoassays, vol 15 (Eds R. H. Burdon and P. H. van Knippenberg, Elsevier, Amsterdam); Zola, 1987, Monoclonal Antibodies: A Manual of Techniques, pp. 147-158 (CRC Press, Inc.); Jalkanen et al., 1985, J. Cell. Biol. 101:976-985; Jalkanen et al., 1987, J. Cell Biol. 105:3087-3096); Harlow and Lane, 1988, Antibodies: A Laboratory Manual, New York: Cold Spring Harbor (ed. 1991 and periodic supplements); John E. Coligan, ed., 1993, Current Protocols In Immunology New York: John Wiley & Sons).

In some embodiments, the in vitro detection is performed on a sample obtained from a subject after administration of a compound that disrupts apical trafficking of a target antigen. Redistribution can be determined by detecting the presence of the target antigen in a sample taken from the subject before and/or after administration of the compound that disrupts apical trafficking. The sample taken from the subject after administration of the compound that disrupts apical trafficking is then assayed for antigen on the basolateral membrane. The sample is, for example, a biopsy sample or other tissue sample. Redistribution of the target antigen is indicative of a disease that can be more effectively treated by disrupting apical targeting of a target antigen and administering an antibody specific for the target antigen rather than treatment of the disease by administering the antibody alone. One such method comprises administering to the subject a compound that disrupts the apical trafficking of the target antigen, removing a sample from the subject, contacting the sample with an antibody specific for the target antigen; and detecting the binding of the antibody to the target antigen.

In other embodiments, a compound that disrupts apical trafficking of a target antigen and an antibody specific for the target antigen is contacted with a sample taken from a subject without prior administration of the compound. Redistribution of a target antigen can be determined by detecting the presence of the target antigen in a sample before administration of the compound that disrupts apical trafficking and comparing it to a sample after administration of the compound that disrupts apical trafficking. One such method comprises contacting the sample with a compound that disrupts the apical trafficking of the target antigen, contacting the sample with an antibody specific for the target antigen, and detecting the binding of the antibody to the target antigen.

The invention provides pharmaceutical compositions. In one embodiment, the pharmaceutical composition comprises a compound that disrupts apical trafficking of a target antigen and an antibody specific for the target antigen. In a preferred embodiment, the pharmaceutical compositions further comprise a pharmaceutically acceptable diluent, carrier, solubilizer, emulsifier, preservative, and/or adjuvant.

In certain embodiments, acceptable formulation materials preferably are nontoxic to recipients at the dosages and concentrations employed. In certain embodiments, the pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, REMINGTON'S PHARMACEUTICAL SCIENCES, 18″ Edition, (A. R. Genrmo, ed.), 1990, Mack Publishing Company.

In certain embodiments, the optimal pharmaceutical composition will be determined by one skilled in the art depending upon, for example, the intended route of administration, delivery format and desired dosage. See, for example, REMINGTON'S PHARMACEUTICAL SCIENCES, supra. In certain embodiments, such compositions may influence the physical state, stability, rate of in vivo release and rate of in vivo clearance of the antibodies of the invention. In certain embodiments, the primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. In preferred embodiments, pharmaceutical compositions comprise Tris buffer of about pH 7.0-8.5, or acetate buffer of about pH 4.0-5.5, and may further include sorbitol or a suitable substitute therefor. In certain embodiments of the invention, the compositions may be prepared for storage by mixing the selected composition having the desired degree of purity with optional formulation agents (REMINGTON'S PHARMACEUTICAL SCIENCES, supra) in the form of a Iyophilized cake or an aqueous solution. Further, in certain embodiments, the compositions may be formulated as a lyophilizate using appropriate excipients such as sucrose.

The pharmaceutical compositions of the invention can be selected for parenteral delivery. Alternatively, the compositions may be selected for inhalation or for delivery through the digestive tract, such as orally. Preparation of such pharmaceutically acceptable compositions is within the skill of the art.

The formulation components are present preferably in concentrations that are acceptable to the site of administration. In certain embodiments, buffers are used to maintain the composition at physiological pH or at a slightly lower pH, typically within a pH range of from about 5 to about 8.

When parenteral administration is contemplated, the pharmaceutical compositions for use in this invention may be provided in the form of a pyrogen-free, parenterally acceptable aqueous solution comprising the compositions and a pharmaceutically acceptable vehicle. A particularly suitable vehicle for parenteral injection is sterile distilled water in which the composition is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that may provide controlled or sustained release of the product which can be delivered via depot injection. In certain embodiments, hyaluronic acid may also be used, having the effect of promoting sustained duration in the circulation. In certain embodiments, implantable drug delivery devices may be used to introduce the pharmaceutical compositions.

Pharmaceutical compositions of the invention can be formulated for inhalation. In these embodiments, the compositions are advantageously formulated as a dry, inhalable powder. In preferred embodiments, the inhalation solutions may also be formulated with a propellant for aerosol delivery. In certain embodiments, solutions may be nebulized. Pulmonary administration and formulation methods therefore are further described in International Patent Application No. PCT/US94/001875, which is incorporated by reference and describes pulmonary delivery of chemically modified proteins. It is also contemplated that formulations can be administered orally. Compositions that are administered in this fashion can be formulated with or without carriers customarily used in the compounding of solid dosage forms such as tablets and capsules. In certain embodiments, a capsule may be designed to release the active portion of the formulation at the point in the gastrointestinal tract when bioavailability is maximized and pre-systemic degradation is minimized. Additional agents can be included to facilitate absorption of the compositions. Diluents, flavorings, low melting point waxes, vegetable oils, lubricants, suspending agents, tablet disintegrating agents, and binders may also be employed.

Additional pharmaceutical compositions will be evident to those skilled in the art, including formulations involving sustained- or controlled-delivery formulations. Techniques for formulating a variety of other sustained- or controlled-delivery means, such as liposome carriers, bio-erodible microparticles or porous beads and depot injections, are also known to those skilled in the art. See, for example, International Patent Application No. PCT/US93/00829, which is incorporated by reference and describes controlled release of porous polymeric microparticles for delivery of pharmaceutical compositions. Sustained-release preparations may include semipermeable polymer matrices in the form of shaped articles, e.g., films, or microcapsules. Sustained release matrices may include polyesters, hydrogels, polylactides (as disclosed in U.S. Pat. No. 3,773,919 and European Patent Application Publication No. EP 058481, each of which is incorporated by reference), copolymers of L-glutamic acid and gamma ethyl-L-glutamate (Sidman et al., 1983, Biopolymers 2:547-556), poly (2-hydroxyethyl-inethacrylate) (Langer et al., 1981, J. Biomed. Mater. Res. 15:167-277 and Langer, 1982, Chem. Tech. 12:98-105), ethylene vinyl acetate (Langer et al., 1981, supra) or poly-D(−)-3-hydroxybutyric acid (European Patent Application Publication No. EP 133,988). Sustained release compositions may also include liposomes that can be prepared by any of several methods known in the art. See, e.g., Eppstein et al., 1985, Proc. Natl. Acad. Sci. U.S.A. 82:3688-3692; European Patent Application Publication Nos. EP 036,676; EP 088,046 and EP 143,949, incorporated by reference.

Pharmaceutical compositions used for in vivo administration are typically provided as sterile preparations. Sterilization can be accomplished by filtration through sterile filtration membranes. When the composition is lyophilized, sterilization using this method may be conducted either prior to or following lyophilization and reconstitution. Compositions for parenteral administration can be stored in lyophilized form or in a solution. Parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, crystal, or as a dehydrated or lyophilized powder. Such formulations may be stored either in a ready-to-use form or in a form (e.g., lyophilized) that is reconstituted prior to administration. The invention also provides kits for producing a single-dose administration unit. The kits of the invention may each contain both a first container having a dried protein and a second container having an aqueous formulation. In certain embodiments of this invention, kits containing single and multi-chambered pre-filled syringes (e.g., liquid syringes and lyosyringes) are provided.

The therapeutically effective amount of compound that disrupts apical trafficking to be employed will depend, for example, upon the therapeutic context and objectives. One skilled in the art will appreciate that the appropriate dosage levels for treatment will vary depending, in part, upon the compound being delivered, the indication for which the compound is being used, the route of administration, and the size (body weight, body surface or organ size) and/or condition (the age and general health) of the subject. In certain embodiments, the clinician may titer the dosage and modify the route of administration to obtain the optimal therapeutic effect. A typical dosage may range from about 10 μg/kg to up to about 1000 mg/kg or more, depending on the factors mentioned above. In preferred embodiments, the dosage may range from 1 mg/kg up to about 500 mg/kg, optionally from 10 mg/kg up to about 100 mg/kg or from 20 mg/kg up to about 50 mg/kg.

The therapeutically effective amount of an antibody-containing pharmaceutical composition to be employed will depend, for example, upon the therapeutic context and objectives. One skilled in the art will appreciate that the appropriate dosage levels for treatment will vary depending, in part, upon the antibody being delivered, the indication for which the antibody is being used, the route of administration, and the size (body weight, body surface or organ size) and/or condition (the age and general health) of the subject. In certain embodiments, the clinician may titer the dosage and modify the route of administration to obtain the optimal therapeutic effect. A typical antibody dosage may range from about 0.1 μg/kg to up to about 30 mg/kg or more, depending on the factors mentioned above. In preferred embodiments, the dosage may range from 0.1 μg/kg up to about 30 mg/kg, optionally from 1 μg/kg up to about 30 mg/kg or from 10 μg/kg up to about 5 mg/kg.

Dosing frequency will depend upon the pharmacokinetic parameters of the particular pharmaceutical composition. Typically, a clinician administers the composition until a dosage is reached that achieves the desired effect. The composition may therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them.

In one embodiment of a method that comprises the administration of both a compound that disrupts the apical trafficking of a target antigen and an antibody, the compound and the antibody are administered at the same time. In another embodiment, the antibody is administered after the administration of the compound. In a further embodiment, the compound is administered after the administration of the antibody but during the time the antibody is still present in the vasculature of the subject.

The route of administration of the pharmaceutical composition is in accord with known methods, e.g., orally, through injection by intravenous, intraperitoneal, intracerebral (intra-parenchymal), intracerebroventricular, intramuscular, intra-ocular, intraarterial, intraportal, or intralesional routes; by sustained release systems or by implantation devices. In certain embodiments, the compositions may be administered by bolus injection or continuously by infusion, or by implantation device.

The composition also may be administered locally via implantation of a membrane, sponge or another appropriate material onto which the desired molecule has been absorbed or encapsulated. In certain embodiments, where an implantation device is used, the device may be implanted into any suitable tissue or organ, and delivery of the desired molecule may be via diffusion, timed-release bolus, or continuous administration.

Another aspect of the invention provides for a kit comprising the pharmaceutical compositions of the invention. In one embodiment, the kit comprises a compound that disrupts apical trafficking of a target antigen and an antibody specific for the target antigen in one unit dose. In another embodiment, the kit comprises a compound that disrupts apical trafficking of a target antigen and an antibody specific for the target antigen in separate doses. In some embodiments, the kit further provides instructions regarding administration of the pharmaceutical compositions.

The foregoing procedures and techniques may be generally performed according to conventional methods well known in the art and as described in various general and more specific references that are cited and discussed throughout the specification. See, e.g., Sambrook et al., 2001, Molecular Cloning: A Laboratoty Manual, 3^{rd} ed., Cold Spring Harbor Laboratory Press, Cold Spring Harbor, N.Y., which is incorporated herein by reference for any purpose. Unless specific definitions are provided, the nomenclature used in connection with, and the laboratory procedures and techniques of, analytical chemistry, organic chemistry, and medicinal and pharmaceutical chemistry described herein are those well known and commonly used in the art. Standard techniques may be used for chemical synthesis, chemical analyses, pharmaceutical preparation, formulation, and delivery and treatment of subjects

All references cited within the body of the instant specification are hereby expressly incorporated by reference in their entirety.

The following examples, including the experiments conducted and the results achieved, are provided for illustrative purposes only and are not to be construed as limiting the invention.

### EXAMPLES

The following examples utilize the MDCK cell culture model. This model is useful in studying trafficking of prostate restricted proteins. Several proteins expressed in prostate gland are similarly targeted in MDCK cells. Information about protein trafficking in MDCK cells can effectively be applied to prostatic epithelial cells, in situ.

Materials and Methods

Cell Culture

MDCK cells (clone II) were obtained from the ATCC (Manassas, Va.) and cultured in DMEM (Gibco BRL, Rockville, Md.) supplemented with 10% FBS, 2 mM L-glutamine, 25 units/ml penicillin, 25 μg/ml streptomycin, and 100 μM non-essential amino acids. Cells were grown at 37° C. in a humidified incubator with 5% CO₂. Cells were treated for 10 hours with 10 mM sodium butyrate to enhance PSMA expression. For experiments involving inhibition of N-glycosylation or microtubule depolymerization, cells were treated for 3 hours with 5 μg/ml tunicamycin (Sigma, St. Louis, Mo.) or 4 μg/ml nocodazole (Sigma), or 2 μM vinblastine, vincristine, or vinorelbine (Sigma) in DMEM at 37° C. prior to the indicated experiment, unless otherwise noted.

DNA Constructs and Transfection

The cDNA encoding full length PSMA (provided by Dr. Warren Heston) was cloned into the pcDNA3 expression vector from Invitrogen, (Carlsbad, Calif.). The sPSMA construct was generated by PCR amplification of codons 53-751 using PSMA cDNA as a template. The 5′ and 3′ primers were used to introduce SfMl and ApaI restriction sites, respectively. This cDNA was cloned into the pSecTag2A vector (Invitrogen) in fusion with an N-terminal sequence encoding the cleavable murine IgKc chain leader sequence for protein secretion. To create the GFP tagged PSMA-Δ103-750 construct, a 309 bp DNA fragment encoding the cytoplasmic, transmembrane, and a 60 amino acid region of the extracellular domain of PSMA was generated by RT-PCR using total RNA isolated from LNCaP cells. The PCR product was digested with Xhol and BamHI and cloned into the pEGFP-N3 expression vector (Clontech, Palo Alto, Calif.). The cDNA encoding the β-subunit of the canine sodium pump (Na,K-ATPase) (provided by Dr. Robert Farley) was PCR amplified and inserted into pEGFP-N3 to create a GFP fusion at the C-terminus (Na,K-β-GFP).

MDCK cells were transfected using calcium phosphate, as previously described (25). Stable clones were selected in 500 μg/ml geneticin (G418, Gibco BRL) for pcDNA3 vectors or 300 μg/ml Zeocin (Invitrogen) for pSecTag2 vector and expression verified by immunofluorescence and immunoblot.

Antibodies

The monoclonal antibody (mAb) J591 against an extracellular epitope of PSMA has been described (26). The mAb 7E11 against an intracellular epitope of PSMA was prepared from hybridoma 7E11 (ATCC, Rockville, Md.). Mouse mAbs raised against Na,K-ATPase α1 (M7-PB-E9) and 1-subunit (M17-P5-F11) have been described (27, 28). Rabbit-anti-mouse (RAM) and mAb against α-tubulin were purchased from Sigma. HRP conjugated goat-anti-mouse IgG was purchased from Transduction Laboratories (Lexington, Ky.). FITC and CY3 conjugated secondary antibodies were purchased from Jackson ImmunoResearch (West Grove, Pa.).

Immunofluoresence and Confocal Microscopy

Tissue sections and MDCK cells were fixed in cold methanol at −20° C. for 30 minutes. Following fixation, specimens were placed in humidified chambers, washed with phosphate buffered saline containing 0.1 mM CaCl₂ and 1 mM MgCl₂ and 0.5% bovine serum albumin (PBS-CM-BSA), incubated 1 hour with primary antibody, washed with PBS-CM-BSA, incubated 30 minutes in secondary antibody, washed with PBS-CM-BSA, rinsed with distilled water, and mounted in Vectashield (Vector, Burlingame, Calif.).

For cell surface staining, MDCK cells were grown on transwell filters and the transepithelial electrical resistance (TER) was determined using an EVOM Epithelial Voltohmeter (World Precision Instruments, Sarasota, Fla.). Values were normalized for filter area after subtracting the background resistance of a filter without cells. TER values of greater than 200Ω/cm² were indicative of TJ formation in MDCK cells (29). Media was removed and replaced with chilled DMEM containing 10 μg/ml J591. Cells were incubated on ice for 30 minutes, rinsed with cold PBS-CM-BSA, fixed in cold methanol and incubated with secondary antibody as described above.

Confocal microscopy was performed using a Fluoview laser scanning confocal microscope (Olympus America, Melville, N.Y.) as described (25). To detect FITC and propidium iodide, samples were excited with krypton and argon lasers and light emitted between 525 and 540 nm was recorded for FITC and above 630 nm for propidium iodide. Images were generated and analyzed using the Fluoview image analysis software, version 2.1.39 (Olympus America).

Cell Surface Biotinylation

MDCK cells were grown to confluence on transwell filters, as determined by TER, and biotinylation of the apical or basolateral surface was performed as described (25). Briefly, 0.5 μg/ml of membrane impermeable EZ-Link Sulfo-NHS-Biotin (Pierce, Rockford, Ill.) in TEA (150 mM NaCl, 10 mM Triethanolamide pH 9.0, 1 mM CaCl₂, 1 mM MgCl₂) was added to either the apical or basolateral chamber. After quenching with 50 mM NH₄Cl in PBS-CM, cells were lysed in 0.5 ml of lysis buffer (150 mM NaCl, 20 mM Tris pH 8, 5 mM EDTA, 1% Triton-X-100, 0.1% BSA, 1 mM PMSF, 5 μg/ml each of antipain, leupeptin, and pepstatin). Total protein from each lysate was used for precipitation (16 hours at 4° C.) with immobilized streptavidin gel (Pierce). Precipitates were washed and prepared for SDS PAGE and immunoblot analysis as described (30).

SDS PAGE and Immunoblot Analysis

Samples were separated on 10% SDS polyacrylamide gels and transferred to nitrocellulose. Membranes were incubated in 5% nonfat milk for 1 hour and immunoblotted with primary antibodies (1:1000) for 2 hours in milk. Membranes were washed three times with PBS with 0.3% Tween-20 (PBST), incubated with horseradish peroxidase-conjugated secondary antibody (1:4000) in milk. After washing three times in PBST and once in PBS, bound antibody was detected by peroxidase-catalyzed ECL-Plus (Amersham, Buckinghamshire, UK). Densitometric analysis and quantification of bands was performed using ImageQuant software (Molecular Dynamics, Sunnyvale, Calif.).

Metabolic Labeling

Confluent monolayers of MDCK-PSMA cells on transwell filters were rinsed twice and incubated for 30 minutes in starving media [cystine/methionine free DMEM (CellGro, Herndon, Va.)] supplemented with 0.2% BSA. Cells were either pulsed for 20 minutes in labeling media [starving media containing 250 μCi/ml Trans[³⁵S]-label (ICN, Costa Mesa, Calif.)] or labeled for 4 hours in the presence or absence of 10 μg/ml of tunicamycin (Sigma). Chase was performed by rinsing filters 3 times with starving media and incubating in DMEM (10% FBS) containing 50 μg/ml of cyclohexamide (Sigma).

Trafficking Assays

For the antibody internalization trafficking assay, MDCK cells were metabolically labeled and chased in DMEM containing 5 μg/ml of mAb J591 added to either the apical or basolateral chamber. Following incubation, cells were rinsed thoroughly with cold PBS-CM. Filters were excised and incubated for >4 hours in lysis buffer at 4° C. Immunocomplexes were precipitated using RAM coated protein A agarose beads. Beads were rinsed and subject to SDS PAGE. Gels were fixed in a solution of 20% methanol and 10% acetic acid, dried, enhanced with salicylic acid, and exposed to film. The relative amount of PSMA for each was calculated as a percentage of the total amount of labeled PSMA precipitated throughout the course of the experiment, as quantified by densitometry.

The biotinylation targeting assay has been previously described (31). Cells were metabolically labeled and chased. Following the indicated time intervals, cells were placed on ice and rinsed three times with cold PBS-CM. Biotinylation of the apical or basolateral surfaces was performed as described above. Filters were excised and incubated for 4 hours in lysis buffer at 4° C. PSMA was immunoprecipitated from cell lysates by incubating with protein A agarose beads coated with RAM and 7E11 for 16 hours at 4° C. Beads were washed and eluted by boiling in 20 μl of 5% SDS. Eluates were removed and resuspended in 1.5 ml lysis buffer. Samples were subsequently incubated with immobilized streptavidin for 16 hours at 4° C. Beads were washed and subject to SDS-PAGE, autoradiography, and densitometry as described above.

Polarized Secretion Assay

MDCK cells expressing sPSMA (MDCK-sPSMA) were grown on transwell filters. Following the establishment of TJs, as assessed by TER, cells were rinsed three times with fresh DMEM and 2.0 ml of fresh media was added to both the apical and basolateral chambers. Cells were incubated for 8 to 10 hours at 37° C., at which point the conditioned media was collected and sPSMA immunoprecipitated using immobilized J591 bound to protein A coated agarose beads. Samples were washed and subject to SDS PAGE, immunoblot analysis, and densitometry as described above.

Post-Golgi Analysis Assays

MDCK-PSMA cells transiently transfected to express Na,K-β-GFP were grown on glass coverslips and treated with nocodazole or tunicamycin. Cells were incubated for 6 hours at 20° C. to accumulate newly synthesized protein in the Golgi and TGN, subsequently transferred to 37° C. for 30 minutes, and subject to immunofluorescence analysis as described above. Laser scanning confocal microscopy was performed using a Zeiss Axiovert 200 inverted microscope (Carl Zeiss, Inc, Thornwood, N.Y.). Samples were excited with Argon and Helium/Neon lasers and single channel images were generated and analyzed using the Zeiss LSM 510 Meta imaging system (Carl Zeiss, Inc.) by recording light emitted between 505 and 543 nm for GFP and above 560 nm for CY3.

Domain Specific Internalization Assay

MDCK-PSMA cells were grown on 0.04 gm pore size polycarbonate transwell filters (Corning, Corning, N.Y.) and grown to confluence as measured by transepithelial electrical resistance (TER) using an EVOM Epithelial Voltometer (World Precision Instruments, Sarasota, Fla.). Values were normalized for the area of the filter after subtracting the background resistance of a filter without cells. TER of 220-250Ω·cm² is indicative of the presence of functional tight junctions (24). Cells were treated with 2 μM of vinblastine, vincristine, or vinorelbine at 37° C. for 3 hours and subsequently incubated at 37° C. for 30 minutes in the presence of the indicated drug and 5 μg/ml of J591 added to either the apical or basolateral chamber. Cells were rinsed in PBS-CM, fixed, and subject to immunofluorescence analysis with FITC-conjugated secondary antibody. Single channel digital microscopic images were collected with an Olympus AX70 upright microscope using identical exposure parameters and analyzed with SPOT imaging software, version 4.0.4 (Diagnostic Instruments, Inc., Sterling Heights, Mich.).

Immunohistochemical Studies

Formalin-fixed, paraffin-embedded tissue samples from patients with metastatic prostate cancer were obtained from the tissue procurement core laboratory (TPCL) at UCLA. Metastatic prostate derived specimens included 4 lesions isolated from lymph nodes and 2 isolated from bone marrow. Serial 5 μm sections were deparaffinized to water and subject to antigen retrieval for 10 minutes at room temperature in 0.05% trypsin or microwaved in citrate buffer. Following antigen retrieval, specimens were incubated in 1% hydrogen peroxide for 10 minutes, blocked with 4% fetal bovine serum in PBS for 1 hour, and incubated with mAb 7E11 (1:50) overnight at 4° C. Samples were subsequently washed and incubated at room temperature with biotinylated goat anti-mouse secondary antibody (Vector) for one hour. Samples were rinsed and subject to A and B reagent. Immunoreactivity was visualized by incubation with diaminobenzidine (DAB) in the presence of hydrogen peroxide. Sections were counterstained with hematoxylin and mounted for microscopic analysis. Control experiments were also performed by incubating tissues with an irrelevant mouse monoclonal IgG.

### Example 1

PSMA is Expressed on the Apical Plasma Membrane of Polarized Epithelial Cells

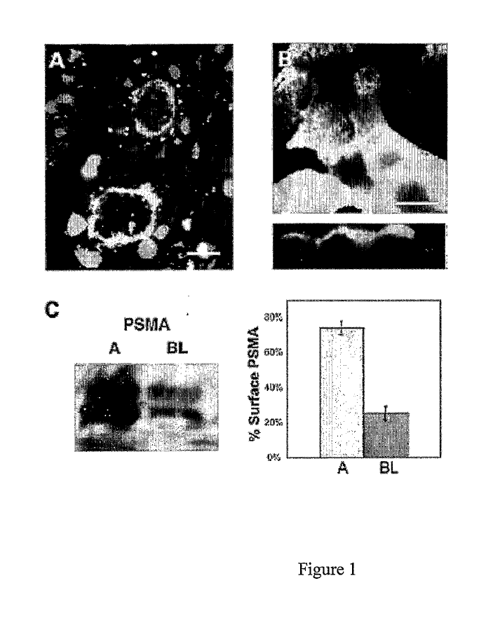

MDCK cell line is a suitable model system to study polarized sorting of prostate restricted transmembrane and secretory proteins (24). Immunofluorescence analysis of tissue sections revealed prominent PSMA localization at the apical plasma membrane of prostatic epithelial cells, with staining at the lumenal interface of the gland (FIG. 1A), recapitulating in situ observations (24). Surface immunofluorescence analysis performed on confluent monolayers of MDCK cells expressing PSMA (MDCK-PSMA) revealed a similar pattern of expression, with PSMA staining localized primarily to the apical membrane in these cells (FIG. 1B). A selective cell surface biotinylation assay was performed in order to provide a quantitative analysis of relative surface PSMA levels. Results of this assay demonstrated that 70 to 79% of PSMA at the cell surface was localized to the apical plasma membrane (FIG. 1C), thus confirming previous results (24).

### Example 2

PSMA is Targeted Directly to the Apical Plasma Membrane

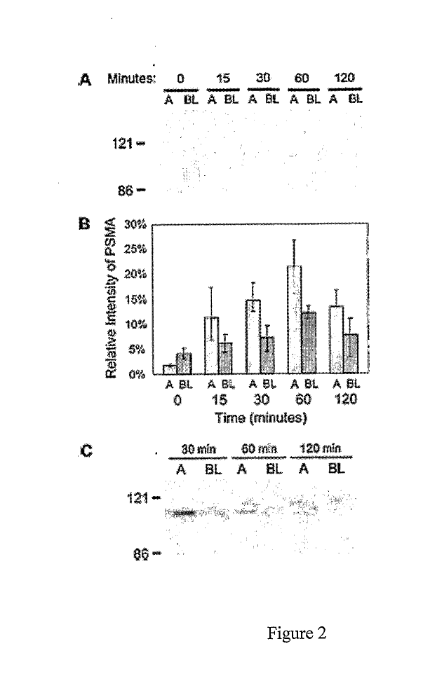

Antibody internalization based trafficking assay was used to determine if apical proteins trafficking involves direct targeting from the TGN or if the proteins are delivered first to the basolateral plasma membrane before undergoing transcytosis to the apical surface. Confluent monolayers of MDCK-PSMA cells grown on transwell filters were metabolically labeled with [³⁵S]-cystine/methionine for a brief pulse and chased in the presence of mAb J591 added to either the apical or basolateral chamber. The J591 mAb recognizes an extracellular epitope on PSMA and is readily internalized by cells expressing PSMA (26). During the period of antibody incubation, PSMA at a particular plasma membrane surface will bind extracellular antibody, even if this localization is transient. Immunocomplexes are then precipitated from cell lysates and subjected to SDS-PAGE and autoradiography. If PSMA were to undergo direct targeting to the apical plasma membrane, a greater amount of metabolically labeled PSMA would be recovered when antibody is added to the apical chamber relative to the basolateral. At each time point subsequent to release, the level of metabolically labeled PSMA immunoprecipitated was between 1.7 and 2.0 fold greater when antibody was added to the apical rather than the basolateral chamber (FIGS. 2A and 2B), thus indicating that while some PSMA may initially be delivered to the basolateral surface, the majority is targeted directly to the apical plasma membrane.

These results were supported by a selective cell surface biotinylation-based targeting assay. Confluent monolayers of MDCK-PSMA cells were metabolically pulsed and chased in normal culture medium before opposing membrane surfaces were labeled with biotin. Autoradiography demonstrated that PSMA is seen predominantly on the apical plasma membrane with a smaller fraction localized to the basolateral surface throughout the course of the experiment (FIG. 2C). The fact that PSMA is primarily observed at the apical plasma membrane even at the initial time point of 30 minutes indicates that the majority of newly synthesized PSMA is targeted to the apical surface.

### Example 3

The Extracellular Domain of PSMA Contains Information for Apical Trafficking

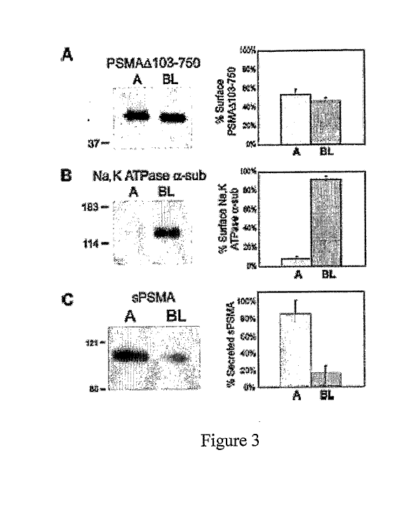

Although signals for apical trafficking may be localized throughout the length of a given transmembrane protein, such signals most commonly reside within the extracellular domain. In order to assess the significance of this domain in apical trafficking, a GFP tagged form of PSMA was created in which the majority of the extracellular domain was removed (PSMA-A103-750). Cell surface biotinylation assays demonstrated that this protein was localized in a non-polarized fashion (FIG. 3A). Immunoblot analysis performed on the same membranes revealed that 90-95% of the α-subunit of the sodium pump (Na,K-ATPase α-sub) was localized at the basolateral surface of these cells (FIG. 3B), demonstrating that the uniform plasma membrane distribution of PSMA is not merely attributable to a general loss of epithelial polarity.

In order to evaluate the trafficking potential offered by the lumenal domain, a secreted form of PSMA (sPSMA) lacking the cytoplasmic and transmembrane domains was created. The sPSMA protein was secreted from MDCK cells as a ˜100 kDa glycoprotein that was recognized by the mAb J591 and that migrated with a molecular mass of ˜80 kDa following treatment with tunicamycin or N-glycosidase (data not shown). A stable MDCK cell line expressing sPSMA (MDCK-sPSMA) was grown to confluence on transwell filters, and the conditioned media was collected from the apical and basolateral chambers. As shown in FIG. 3C, sPSMA was secreted almost exclusively from the apical plasma membrane, further implicating the existence of a targeting signal encoded within the extracellular domain of PSMA.

### Example 4

Apical Trafficking of PSMA Requires N-glycosylation

The extracellular domain of PSMA is highly glycosylated, with approximately 25% of the mass of PSMA attributable to N-linked carbohydrates (32). Given the significance of oligosaccharide moieties in apical trafficking, we investigated the role of N-glycosylation in trafficking of PSMA (14, 33).

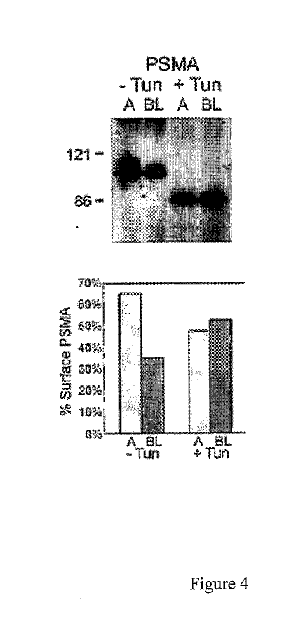

Confluent monolayers of MDCK-PSMA cells were metabolically labeled in the presence or absence of tunicamycin. This drug prevents N-glycosylation in the endoplasmic reticulum, and has been used extensively to assess the role of glycosylation in protein trafficking (34, 35). Selective biotinylation of the apical or basolateral plasma membrane revealed that while the majority of surface PSMA is normally localized to the apical plasma membrane, inhibition of N-glycosylation abolished the polarized expression of PSMA and resulted in equivalent levels at both plasma membrane surfaces (FIG. 4).

Inhibition of N-glycosylation also resulted in a dramatic alteration in PSMA localization within post-Golgi transport vesicles. Incubation of MDCK-PSMA cells at 20° C. was used to inhibit post-Golgi transit and accumulate proteins within the TGN (36). Both PSMA and a GFP tagged version of the basolaterally targeted Na,K-ATPase-subunit (Na,K-β-GFP) were localized to the TGN following incubation at 20° C. (data not shown). These cells were subsequently transferred to 37° C., allowing proteins to exit from the TGN. In the absence of tunicamycin, PSMA and Na,K-ATPase localized to distinct post-Golgi vesicles in regions proximal to the TGN. However, the level of co-localization of vesicles containing these markers increased to approximately 43% (38/88) when cells are incubated with tunicamycin, indicating a role for N-glycosylation PSMA sorting into distinct post-Golgi vesicles (data not shown).

### Example 5

Microtubules are Necessary for Apical Trafficking of PSMA

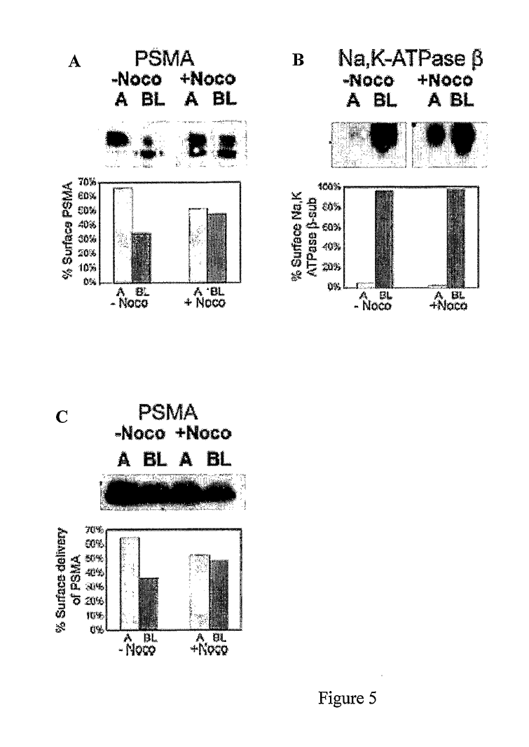

The integrity of the microtubule cytoskeleton is essential for the targeted delivery of many apical proteins in polarized epithelial cells (37, 38). To address the significance of microtubules in PSMA trafficking, MDCK-PSMA cells were treated with the microtubule-depolymerizing agent, nocodazole. As shown in FIGS. 5A-C, nocodazole treatment resulted in a dramatic redistribution of PSMA. Surface immunofluoresence revealed increased PSMA expression at the basolateral plasma membrane relative to untreated cells (data not shown). These data were also confirmed by cell surface biotinylation experiments, which demonstrate a homogeneous distribution of PSMA at both plasma membrane domains following nocodazole treatment (FIG. 5A). Polarity of the basolateral marker Na,K-ATPase was unaffected by nocodazole treatment, confirming the conservation of TJ integrity and epithelial polarity in these cells (FIG. 5B).

While tunicamycin and nocodazole treatment both resulted in a loss of PSMA polarity, the localization of PSMA within post-Golgi vesicles after treatment with these drugs was distinctly different. Following release from a 20° C. block, PSMA and Na,K-β-GFP did not show an increased co-localization in the presence of nocodazole (data not shown). These results indicate that trafficking of PSMA into distinct post-Golgi vesicles was unaffected by microtubule depolymerization.

While microtubule depolymerization does not impact the sorting of PSMA into post-Golgi vesicles, the delivery of these vesicles to the plasma membrane fails to occur in a polarized manner. Confluent monolayers of MDCK-PSMA cells on transwell filters were pulsed and chased in the presence of extracellular mAb J591. In the absence of nocodazole, approximately 1.9 fold more radiolabeled PSMA was precipitated when J591 was added to the apical chamber compared to the basolateral, consistent with our earlier findings (FIG. 5C). However, in the presence of nocodazole, equivalent levels of radiolabeled PSMA were precipitated regardless of the chamber to which J591 was added, thus demonstrating that newly synthesized PSMA was delivered in a non-polarized fashion (FIG. 5C). These results suggest that microtubule integrity is necessary for proper delivery and retention of PSMA at the plasma membrane domain.

### Example 6

Vinca Alkaloids Promote mAb J591 Uptake from the Basolateral Plasma Membrane

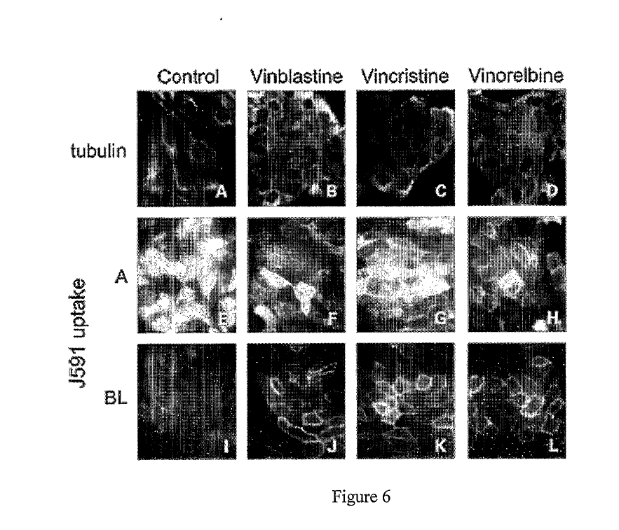

The vinca alkaloids are a class of drugs that inhibit microtubule assembly and are used in the treatment of a number of malignant diseases, including prostate cancer. Treatment of MDCK-PSMA cells with vinblastine, vincristine, or vinorelbine was sufficient to induce extensive depolymerization of the microtubule cytoskeleton (FIG. 6A-D). Confluent monolayers of MDCK-PSMA cells were subjected to J591 internalization assays in order to determine how vinca alkaloid treatment influences PSMA localization. While polarized monolayers of untreated MDCK-PSMA cells readily internalized mAb J591 added to the apical chamber (FIG. 6E), very little antibody was internalized from the basolateral surface (FIG. 61). Following treatment with vinca alkaloids, J591 was also taken up from the apical surface, albeit at decreased levels relative to untreated cells (FIG. 6F-H), however, these cells exhibited a dramatic increase in J591 internalization from the basolateral surface (FIG. 6J-L).

### Example 7

Polarized Morphology of Prostate Tumor Cells

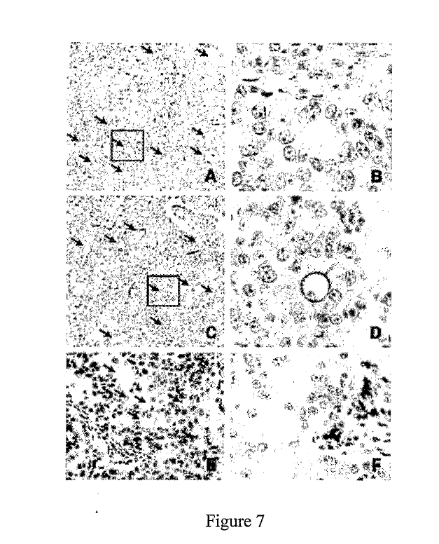

Histological assessment of a metastatic lesion from lymph node demonstrates diffused prostate tumor infiltration replacing the lymph node parenchyma (FIG. 7A). The enlarged tumor cells contain large and prominent nuclei and mitotic figures are readily observed. The prostate cancer cells form sheets with several areas of glandular differentiation. These glandular structures have clearly identifiable lumenal spaces occasionally containing pink secretions. The tumor cells surrounding the lumenal spaces show similar morphology to that seen in well-differentiated primary adenocarcinoma of the prostate with distinct plasma membrane organization (FIGS. 7A and B). Immunohistochemical analysis revealed that these cells express PSMA, and that this antigen is restricted to the apical surface facing the lumen (FIGS. 7C and D). This staining was clearly distinct from that of the endothelial cell marker CD 34 and CD31. Antibodies to these antigens stained small vessels but not the glandular structures, thus excluding the possibility that these PSMA expressing structures are actually blood vessels (data not shown). These results indicate that prostatic carcinoma cells may retain a well-differentiated morphology, even following metastasis to distal sites.

### REFERENCES

1. Silver, D. A., Pellicer, I., Fair, W. R., Heston, W. D., and Cordon-Cardo, C. Prostate-specific membrane antigen expression in normal and malignant human tissues. Clin Cancer Res, 3: 81-85, 1997.
2. Sweat, S. D., Pacelli, A., Murphy, G. P., and Bostwick, D. G. Prostate-specific membrane antigen expression is greatest in prostate adenocarcinoma and lymph node metastases. Urology, 52: 637-640, 1998.
3. Wright, G. L., Jr., Grob, B. M., Haley, C., Grossman, K., Newhall, K., Petrylak, D., Troyer, J., Konchuba, A., Schellhammer, P. F., and Moriarty, R. Upregulation of prostate-specific membrane antigen after androgen-deprivation therapy. Urology, 48: 326-334, 1996.
4. Ross, J. S., Sheehan, C. E., Fisher, H. A., Kaufman, R. P., Jr., Kaur, P., Gray, K., Webb, I., Gray, G. S., Mosher, R., and Kallakury, B. V. Correlation of primary tumor prostate-specific membrane antigen expression with disease recurrence in prostate cancer. Clin Cancer Res, 9: 6357-6362, 2003.
5. Chang, S. S, and Heston, W. D. The clinical role of prostate-specific membrane antigen (PSMA). Urol Oncol, 7: 7-12, 2002.
6. Christiansen, J. J. and Rajasekaran, A. K. Biological impediments to monoclonal antibody based immunotherapy. Mol Cancer Ther, 2004.
7. Rodriguez-Boulan, E., Salas, P. J., Sargiacomo, M., Lisanti, M., Lebivic, A., Sambuy, Y., Vega-Salas, D., and Graeve, L. Methods to estimate the polarized distribution of surface antigens in cultured epithelial cells. Methods Cell Biol, 32: 37-56, 1989.
8. Anderson, J. M. and Van Itallie, C. M. Tight junctions and the molecular basis for regulation of paracellular permeability. Am J Physiol, 269: G467-475, 1995.
9. van Meer, G. and Simons, K. The function of tight junctions in maintaining differences in lipid composition between the apical and the basolateral cell surface domains of MDCK cells. Embo J, 5:1455-1464, 1986.
10. Simons, K. and Wandinger-Ness, A. Polarized sorting in epithelia. Cell, 62: 207-210, 1990.
11. Apodaca, G., Bomsel, M., Arden, J., Breiffeld, P. P., Tang, K., and Mostov, K. E. The polymeric immunoglobulin receptor. A model protein to study transcytosis. J Clin Invest, 87: 1877-1882, 1991.
12. Hunziker, W. and Furney, C. A di-leucine motif mediates endocytosis and basolateral sorting of macrophage IgG Fc receptors in MDCK cells. Embo J, 13: 2963-2967, 1994.
13. Honing, S, and Hunziker, W. Cytoplasmic determinants involved in direct lysosomal sorting, endocytosis, and basolateral targeting of rat Igp120 (lamp-I) in MDCK cells. J Cell Biol, 128: 321-332, 1995.
14. Scheiffele, P., Peranen, J., and Simons, K. N-glycans as apical sorting signals in epithelial cells. Nature, 378: 96-98, 1995.
15. Yeaman, C., Le Gall, A. H., Baldwin, A. N., Monlauzeur, L., Le Bivic, A., and Rodriguez-Boulan, E. The O-glycosylated stalk domain is required for apical sorting of neurotrophin receptors in polarized MDCK cells. J Cell Biol, 139: 929-940, 1997.
16. Moyer, B. D., Denton, J., Karlson, K. H., Reynolds, D., Wang, S., Mickle, J. E., Milewski, M., Cutting, G. R., Guggino, W. B., Li, M., and Stanton, B. A. A PDZ-interacting domain in CFTR is an apical membrane polarization signal. J Clin Invest, 104: 1353-1361, 1999.
17. Brown, D. A. and Rose, J. K. Sorting of GPI-anchored proteins to glycolipid-enriched membrane subdomains during transport to the apical cell surface. Cell, 68: 533-544, 1992.
18. Barman, S, and Nayak, D. P. Analysis of the transmembrane domain of influenza virus neuraminidase, a type II transmembrane glycoprotein, for apical sorting and raft association. J Virol, 74: 6538-6545, 2000.
19. Chuang, J. Z. and Sung, C. H. The cytoplasmic tail of rhodopsin acts as a novel apical sorting signal in polarized MDCK cells. J Cell Biol, 142: 1245-1256, 1998.
20. Bacallao, R., Antony, C., Dotti, C., Karsenti, E., Stelzer, E. H., and Simons, K. The subcellular organization of Madin-Darby canine kidney cells during the formation of a polarized epithelium. J Cell Biol, 109: 2817-2832, 1989.
21. Zegers, M. M., Zaal, K. J., van, I. S.C., Klappe, K., and Hoekstra, D. Actin filaments and microtubules are involved in different membrane traffic pathways that transport sphingolipids to the apical surface of polarized HepG2 cells. Mol Biol Cell, 9: 1939-1949, 1998.
22. Tai, A. W., Chuang, J. Z., and Sung, C. H. Cytoplasmic dynein regulation by subunit heterogeneity and its role in apical transport. J Cell Biol, 153: 1499-1509, 2001.
23. Breiffeld, P. P., McKinnon, W. C., and Mostov, K. E. Effect of nocodazole on vesicular traffic to the apical and basolateral surfaces of polarized MDCK cells. J Cell Biol, 111: 2365-2373, 1990.
24. Christiansen, J. J., Rajasekaran, S. A., Moy, P., Butch, A., Goodglick, L., Gu, Z., Reiter, R. E., Bander, N. H., and Rajasekaran, A. K. Polarity of prostate specific membrane antigen, prostate stem cell antigen, and prostate specific antigen in prostate tissue and in a cultured epithelial cell line. Prostate, 55: 9-19, 2003.
25. Rajasekaran, S. A., Palmer, L. G., Quan, K., Harper, J. F., Ball, W. J., Jr., Bander, N. H., Peralta Soler, A., and Rajasekaran, A. K. Na,K-ATPase beta-subunit is required for epithelial polarization, suppression of invasion, and cell motility. Mol Biol Cell, 12: 279-295, 2001.
26. Liu, H., Rajasekaran, A. K., Moy, P., Xia, Y., Kim, S., Navarro, V., Rahmati, R., and Bander, N. H. Constitutive and antibody-induced internalization of prostate-specific membrane antigen. Cancer Res, 58: 4055-4060, 1998.
27. Sun, Y. and Ball, W. J., Jr. Identification of antigenic sites on the Na⁺/K(+)-ATPase beta-subunit: their sequences and the effects of thiol reduction upon their structure. Biochim Biophys Acta, 1207: 236-248, 1994.
28. Abbott, A. and Ball, W. J., Jr. The epitope for the inhibitory antibody M7-PB-E9 contains Ser-646 and Asp-652 of the sheep Na⁺,K(+)-ATPase alpha-subunit. Biochemistry, 32: 3511-3518, 1993.
29. Gonzalez-Mariscal, L., Chavez de Ramirez, B., and Cereijido, M. Tight junction formation in cultured epithelial cells (MDCK). J Membr Biol, 86: 113-125, 1985.
30. Rajasekaran, A. K., Hojo, M., Huima, T., and Rodriguez-Boulan, E. Catenins and zonula occludens-1 form a complex during early stages in the assembly of tight junctions. J Cell Biol, 132: 451-463, 1996.
31. Le Bivic, A., Real, F. X., and Rodriguez-Boulan, E. Vectorial targeting of apical and basolateral plasma membrane proteins in a human adenocarcinoma epithelial cell line. Proc Natl Acad Sci USA, 86: 9313-9317, 1989.
32. Holmes, E. H., Greene, T. G., Tino, W. T., Boynton, A. L., Aldape, H. C., Misrock, S. L., and Murphy, G. P. Analysis of glycosylation of prostate-specific membrane antigen derived from LNCaP cells, prostatic carcinoma tumors, and serum from prostate cancer patients. Prostate Suppl, 7: 25-29, 1996.
33. Su, T., Cariappa, R., and Stanley, K. N-glycans are not a universal signal for apical sorting of secretory proteins. FEBS Lett, 453: 391-394, 1999.
34. Gut, A., Kappeler, F., Hyka, N., Balda, M. S., Hauri, H. P., and Matter, K. Carbohydrate-mediated Golgi to cell surface transport and apical targeting of membrane proteins. Embo J, 17: 1919-1929, 1998.
35. Marmorstein, A. D., Csaky, K. G., Baffi, J., Lam, L., Rahaal, F., and Rodriguez-Boulan, E. Saturation of, and competition for entry into, the apical secretory pathway. Proc Natl Acad Sci USA, 97: 3248-3253, 2000.
36. Griffiths, G., Fuller, S. D., Back, R., Hollinshead, M., Pfeiffer, S., and Simons, K. The dynamic nature of the Golgi complex. J Cell Biol, 108: 277-297, 1989.
37. Kreitzer, G., Schmoranzer, J., Low, S. H., Li, X., Gan, Y., Weimbs, T., Simon, S. M., and Rodriguez-Boulan, E. Three-dimensional analysis of post-Golgi carrier exocytosis in epithelial cells. Nat Cell Biol, 5: 126-136, 2003.
38. Saunders, C. and Limbird, L. E. Disruption of microtubules reveals two independent apical targeting mechanisms for G-protein-coupled receptors in polarized renal epithelial cells. J Biol Chem, 272:19035-19045, 1997.
39. Ballangrud, A. M., Yang, W. H., Charlton, D. E., McDevitt, M. R., Hamacher, K. A., Panageas, K. S., Ma, D., Bander, N. H., Scheinberg, D. A., and Sgouros, G. Response of LNCaP spheroids after treatment with an alpha-particle emitter (213Bi)-labeled anti-prostate-specific membrane antigen antibody (J591). Cancer Res, 61: 2008-2014, 2001.
40. McDevitt, M. R., Barendswaard, E., Ma, D., Lai, L., Curcio, M. J., Sgouros, G., Ballangrud, A. M., Yang, W. H., Finn, R. D., Pellegrini, V., Geerlings, M. W., Jr., Lee, M., Brechbiel, M. W., Bander, N. H., Cordon-Cardo, C., and Scheinberg, D. A. An alpha-particle emitting antibody ([213Bi]J591) for radioimmunotherapy of prostate cancer. Cancer Res, 60: 6095-6100, 2000.
41. Bander, N. H., Trabulsi, E. J., Kostakoglu, L., Yao, D., Vallabhajosula, S., Smith-Jones, P., Joyce, M. A., Milowsky, M., Nanus, D. M., and Goldsmith, S. J. Targeting metastatic prostate cancer with radiolabeled monoclonal antibody J591 to the extracellular domain of prostate specific membrane antigen. J Urol, 170: 1717-1721, 2003.
42. Countouriotis, A., Moore, T. B., and Sakamoto, K. M. Cell surface antigen and molecular targeting in the treatment of hematologic malignancies. Stem Cells, 20: 215-229, 2002.
43. Khawli, L. A., Miller, G. K., and Epstein, A. L. Effect of seven new vasoactive immunoconjugates on the enhancement of monoclonal antibody uptake in tumors. Cancer, 73: 824-831, 1994.
44. Epenetos, A. A., Snook, D., Durbin, H., Johnson, P. M., and Taylor-Papadimitriou, J. Limitations of radiolabeled monoclonal antibodies for localization of human neoplasms. Cancer Res, 46: 3183-3191, 1986.
45. Au, J. L., Jang, S. H., Zheng, J., Chen, C. T., Song, S., Hu, L., and Wientjes, M. G. Determinants of drug delivery and transport to solid tumors. J Control Release, 74: 31-46, 2001.
46. Jain, R. K. Vascular and interstitial barriers to delivery of therapeutic agents in tumors. Cancer Metastasis Rev, 9: 253-266, 1990.
47. Ahnen, D. J., Nakane, P. K., and Brown, W. R. Ultrastructural localization of carcinoembryonic antigen in normal intestine and colon cancer: abnormal distribution of CEA on the surfaces of colon cancer cells. Cancer, 49: 2077-2090, 1982.
48. Tobioka, H., Isomura, H., Kokai, Y., and Sawada, N. Polarized distribution of carcinoembryonic antigen is associated with a tight junction molecule in human colorectal adenocarcinoma. J Pathol, 198: 207-212, 2002.
49. Delaloye, B., Bischof-Delaloye, A., Buchegger, F., von Fliedner, V., Grob, J. P., Volant, J. C., Pettavel, J., and Mach, J. P. Detection of colorectal carcinoma by emission-computerized tomography after injection of 1231-labeled Fab or F(ab′)2 fragments from monoclonal anti-carcinoembryonic antigen antibodies. J Clin Invest, 77: 301-311, 1986.
50. Mach, J. P., Buchegger, F., Formi, M., Ritschard, J., Carrel, S., Egley, R., Donath, A., and Rohner, A. Immunoscintigraphy for the detection of human carcinoma after injection of radiolabeled monoclonal anti-carcinoembryonic antigen antibodies. Curr Top Microbiol Immunol, 104: 49-55, 1983.
51. McIntosh, D. P., Tan, X. Y., Oh, P., and Schnitzer, J. E. Targeting endothelium and its dynamic caveolae for tissue-specific transcytosis in vivo: a pathway to overcome cell barriers to drug and gene delivery. Proc Natl Acad Sci USA, 99: 1996-2001, 2002.
52. Curnis, F., Sacchi, A., and Corti, A. Improving chemotherapeutic drug penetration in tumors by vascular targeting and barrier alteration. J Clin Invest, 110: 475-482, 2002.
53. Christiansen, J. J. et al., N-glycosylation and microtubule integrity are involved in apical targeting of prostate-specific membrane antigen: implications for immunotherapy. Mol Cancer Ther, 4:704-14, 2005.

What is claimed is:

1. A method treatment of a cellular proliferative disease in a subject, wherein said disease is characterized by the presence of a target antigen with apical polarity, said method comprising administering to the subject:
a. a compound that disrupts the apical trafficking of the target antigen; and
b. an antibody specific for the target antigen.

2. The method of claim 1, wherein said proliferative disease is a cancer.

3. The method of claim 2, wherein said cancer is prostrate cancer.

4. The method of claim 1, wherein said target antigen is PSMA.

5. The method of claim 1, wherein said antibody is specific for PSMA.

6. The method of claim 5, wherein said antibody is selected from the group consisting of 7E11, J591 and PM2J004.5.

7. The method of claim 1, wherein said compound disrupts microtubule integrity.

8. The method of claim 7, wherein said compound is a vinca alkaloid.

9. The method of claim 8, wherein said vinca alkaloid is selected from the group consisting of vinblastine, vincristine, vindesine and vinorelbine.

10. The method of claim 1, wherein said compound interferes with N-glycosylation of the target antigen.

11. The method of claim 10, wherein said compound is selected from the group consisting of tunicamycin, swainsonine, and deoxymannojirmycin.

12. The method of claim 11, wherein said antibody further comprises an effector group.

13. The method of claim 12, wherein said effector group is a cytotoxic agent.

14. The method of claim 13, wherein said cytotoxic agent is selected from the group consisting of radioisotopes, radionuclides, and chemotherapeutic agents.

15. A method of detecting the presence of a cellular proliferative disease in a subject, wherein said disease is characterized by the presence of a target antigen with apical polarity, said method comprising:
a. administering to the subject a compound that disrupts the apical trafficking of the target antigen;
b. administering to the subject an antibody specific for the target antigen; and
c. detecting the binding of the antibody to the target antigen.

16. The method of claim 15, wherein said proliferative disease is a cancer.

17. The method of claim 16, wherein said cancer is prostrate cancer.

18. The method of claim 15, wherein said target antigen is PSMA.

19. The method of claim 15, wherein said antibody is specific for PSMA.

20. The method of claim 19, wherein said antibody is selected from the group consisting of 7E11, J591 and PM2J004.5.

21. The method of claim 15, wherein said compound disrupts microtubule integrity.

22. The method of claim 21, wherein said compound is a vinca alkaloid.

23. The method of claim 22 wherein said vinca alkaloid is selected from the group consisting of vinblastine, vincristine, vindesine and vinorelbine.

24. The method of claim 15, wherein said compound interferes with N-glycosylation of the target antigen.

25. The method of claim 24, wherein said compound is tunicamycin.

26. The method of claim 15, wherein said antibody further comprises a detectable label.

27. The method of claim 26, wherein said detectable label is selected from the group consisting of radioisotopes, radionuclides, fluorescent groups, paramagnetic groups, enzymatic groups, chemiluminescent groups, biotinyl groups, or predetermined polypeptide epitopes recognized by a secondary reporter.

28. A method of detecting the presence of a cellular proliferative disease in a sample, wherein said disease is characterized by the presence of a target antigen with apical polarity, said method comprising:
a. contacting the basolateral membrane of the sample with a compound that disrupts the apical trafficking of the target antigen;
b. contacting the sample with an antibody specific for the target antigen; and
c. detecting the binding of the antibody to the target antigen on the basolateral membrane.

29. A method of detecting the presence of a cellular proliferative disease in a subject, wherein said disease is characterized by the presence of a target antigen with apical polarity, said method comprising:
a. administering to the subject a compound that disrupts the apical trafficking of the target antigen;
b. removing a sample from the subject;
c. contacting basolateral membrane of the sample with an antibody specific for the target antigen; and
d. detecting the binding of the antibody to the target antigen on the basolateral membrane.
